(19) United States
(12) Patent Application Publication
(10) Pub. No.: US 2025/0201401 A1
(43) Pub. Date: Jun. 19, 2025

(54) SELECTING, QUEUING, AND DISPATCHING REMOTELY LOCATED PATIENT SERVICE PROVIDERS

(71) Applicant: West Affum Holdings DAC, Dublin (IE)

(72) Inventor: Brian D. Webster, Mercer Island, WA (US)

(73) Assignee: West Affum Holdings DAC, Dublin (IE)

(21) Appl. No.: 19/063,897

(22) Filed: Feb. 26, 2025

Related U.S. Application Data
(63) Continuation of application No. 17/118,294, filed on Dec. 10, 2020.
(60) Provisional application No. 62/991,552, filed on Mar. 18, 2020.

(51) Int. Cl. G16H 40/67; A61N 1/04; A61N 1/39; G06Q 10/1093; G16H 10/60; G16H 40/20; G16H 80/00

(52) U.S. Cl. CPC G16H 40/67; A61N 1/0484; A61N 1/3904; G06Q 10/1095; G16H 10/60; G16H 40/20; G16H 80/00

(57) ABSTRACT

Systems are provided to perform automated identification and dispatching of resources (e.g., patient service responders) to provide individualization of medical products and service to specific patients. Such systems can use decision-based tree methods. In some examples, the systems can learn from historical data (using AI technology, for example) to autonomously match patient's medical and personal needs and preferences with responder options and the requisite inventory. Such systems can be scalable for a growing number of patients and responders and can improve patient quality of care, treatment, and satisfaction. The automated systems and methods can alleviate burdens on medical network and resources including reduction of redundancies and costs. It can also help reduce, and if needed, quickly address any errors, human and/or technical, and aid with quality assurance.

### CROSS REFERENCE TO RELATED APPLICATIONS

This application is a continuation of U.S. patent application Ser. No. 17/118,294, filed on Dec. 10, 2020, titled “SELECTING, QUEUING, AND DISPATCHING REMOTELY LOCATED PATIENT SERVICE PROVIDERS,” which claims the benefit of and priority to U.S. Provisional Patent Application Ser. No. 62/991,552, filed on Mar. 18, 2020, titled “SYSTEM AND METHOD SYSTEM AND METHOD FOR SELECTING, QUEUING, AND DISPATCHING REMOTELY LOCATED PRODUCT SERVICE PROVIDERS,” the disclosures of which are hereby incorporated by reference for all purposes.

### TECHNICAL FIELD

The disclosed subject matter pertains generally to the area of individualized medical products, and more specifically to systems for managing individualization of those medical products to specific patients.

### BACKGROUND INFORMATION

When people suffer from some types of heart arrhythmias, the result may be that blood flow to various parts of the body is reduced. Some arrhythmias may even result in a Sudden Cardiac Arrest (SCA). SCA can lead to death very quickly, e.g., within 10 minutes, unless treated in the interim. Some observers have thought that SCA is the same as a heart attack, which it is not.

Some people have an increased risk of SCA. Such people include patients who have had a heart attack, or a prior SCA episode. A frequent recommendation for these people is to receive an Implantable Cardioverter Defibrillator (ICD). The ICD is surgically implanted in the chest, and continuously monitors the patient's electrocardiogram (ECG). If certain types of heart arrhythmias are detected, then the ICD delivers an electric shock through the heart.

In some circumstances, those identified as candidates for an ICD are unable to move forward with the surgery, such as while recovering from some other cardiac event. Those candidates may be given a Wearable Cardioverter Defibrillator (WCD) system to wear until the ICD is implanted. WCD systems have also been called wearable cardiac defibrillator systems. A WCD system typically includes a harness, vest, belt, or other garment that the patient is to wear. The WCD system further includes electronic components, such as a defibrillator and electrodes, coupled to the harness, vest, or other garment. When the WCD system is properly worn, the electrodes make good electrical contact with the patient's skin, and therefore can help sense the patient's ECG. If a shockable heart arrhythmia is detected from the ECG, then the defibrillator delivers an appropriate electric shock through the patient's body, and thus through the heart. This may restart the patient's heart and thus save their life.

One problem with a Wearable Cardioverter Defibrillator (WCD), when initially prescribed to a patient, is that if it is uncomfortable or poorly fitted to the patient's body, the electrodes may not make good contact which may result in skewed data acquisition, noise, improper monitoring, and potentially even unnecessary therapy. A poorly fitted WCD may also irritate the patient and compromise wear compliance. It is therefore advantageous that skilled and trained responders provide initial and continuing interaction with the patient to help ensure that the fit of the WCD is proper and well-adjusted, and that it does not cause the patient unnecessary discomfort or frustration. However, dispatching the appropriate trained responders when needed to the appropriate location and with the appropriate equipment poses a logistical difficulty.

The subject matter discussed in this Background section is not necessarily prior art to the disclosure and should not be presumed so simply because it is presented in this section. Rather, the discussion of any subject matter in this Background section should be read in conjunction with the detailed description below as merely a full and complete disclosure of the novel concepts.

### SUMMARY OF THE DISCLOSURE

Embodiments of the present disclosure provide an automated system that can effectively streamline localization, screening, selection and queuing of skilled trained responders who can perform the customized fitting or adjustments, within a predetermined geographic perimeter, and who, when needed, also have access to the requisite product inventory. Various embodiments systems, as described herein, perform automated identification and dispatching of resources (e.g., patient service responders) to provide individualization of medical products and service to specific patients. Such systems can use decision-based tree methods. In some embodiments, the systems can learn from historical data (using AI technology, for example) to autonomously match patient's medical and personal needs and preferences with responder options and the requisite inventory. Such systems can be scalable for a growing number of patients and responders and can improve patient quality of care, treatment, and satisfaction. The automated systems and methods, as disclosed herein, can alleviate burdens on medical network and resources including reduction of redundancies and costs. It can also help reduce, and if needed, quickly address any errors, human and/or technical, and aid with quality assurance.

### BRIEF DESCRIPTION OF THE DRAWINGS

FIG. 1 is a conceptual diagram generally showing components of a medical device that may be adapted to implement embodiments of this disclosure.

FIG. 2 is another functional diagram showing components of an external defibrillator, made according to embodiments.

FIG. 3 is another functional diagram illustrative sample embodiments of components of an illustrative WCD system.

FIG. 4 is a conceptual diagram illustrating how multiple electrodes of a WCD system may be used for sensing ECG signals along different vectors according to embodiments.

FIG. 5 is a functional block diagram generally illustrating components of one illustrative Resource Management System that implements embodiments of the disclosure.

FIG. 6 illustrates an illustrative process that may be performed by one or more of the components of the Resource Management System to implement the teachings of this disclosure.

### DETAILED DESCRIPTION OF EMBODIMENTS

Briefly stated, this disclosure describes embodiments of a system for efficiently managing resources of a medical device system. More specifically, disclosed are systems and methods for dispatching responders to assist a patient with the fitment of a wearable medical device. Generally stated, the several embodiments perform actions and analyses to identify one or more appropriate Patient Service Responders (PSRs). These PSRs assist patients with a medical product fitting, adjusting, positioning, training, and/or any additional follow-ups. In some embodiments, these PSRs are qualified to provide such services, such as by receiving training and periodic renewals of such qualification. In one example, a patient is prescribed a wearable cardioverter defibrillator and needs a skilled professional to perform a fitting.

This disclosure is structured as follows: First will be described implementations of a Wearable Cardioverter Defibrillator of the type that may benefit from embodiments of the entire disclosure. Next will be described specific implementations of a Resource Management System that may implement embodiments of the teachings of the disclosure. Finally will be described specific implementations of a process used by a resource management system to implement the teachings of this disclosure.

### EMBODIMENTS OF A WEARABLE MEDICAL DEVICE

A wearable cardioverter defibrillator (WCD) system that may be used in various embodiments protects an ambulatory patient by electrically restarting their heart if needed. Such a WCD system may have a number of components. These components can be provided separately as modules that can be interconnected, or can be combined with other components, and so on.

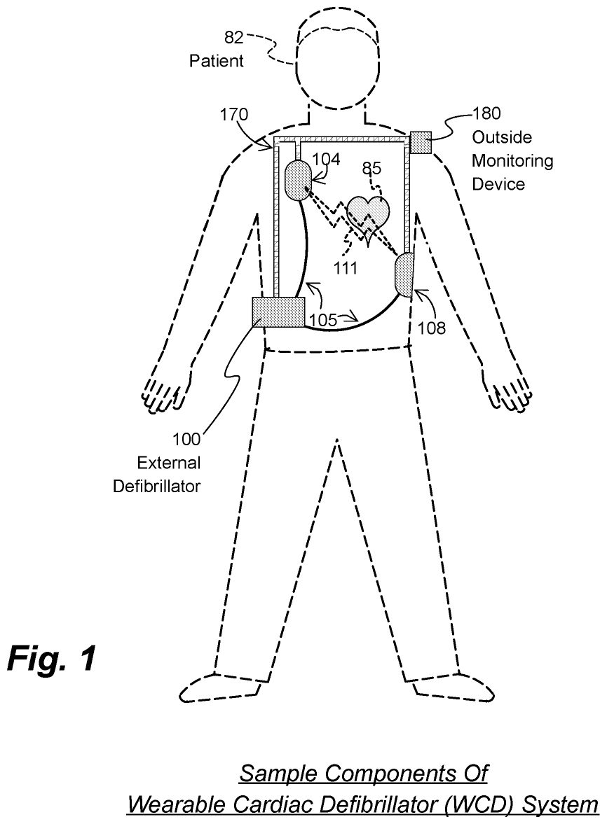

FIG. 1 is a conceptual diagram generally showing components of a medical device that may be adapted to implement embodiments of this disclosure. In this particular example, the medical device is a wearable cardioverter defibrillator (WCD) system. In alternative embodiments, the medical device may be any other wearable medical system, such as, for example, a patient monitoring system. Such a patient monitoring system may include some, but not all of the components of a WCD. Similarly, the patient monitoring system may include components in addition to those shown for the illustrative WCD shown in FIG. 1. Many other alternatives will become apparent to those skilled in the art upon reading the teachings of this disclosure.

As illustrated, FIG. 1 depicts a patient 82. Patient 82 may also be referred to as a person and/or wearer, since the patient is wearing components of the WCD system. Patient 82 is ambulatory, which means that, while wearing the wearable portion of the WCD system, patient 82 can walk around and is not necessarily bedridden. While patient 82 may be considered to be also a “user” of the WCD system, this is not a requirement. For instance, a user of the WCD may also be a clinician such as a doctor, nurse, emergency medical technician (EMT) or other similarly tasked individual or group of individuals. In some cases, a user may even be a bystander. The particular context of these and other related terms within this description should be interpreted accordingly.

A WCD system according to embodiments can be configured to defibrillate the patient who is wearing the designated parts the WCD system. Defibrillating can be by the WCD system delivering an electrical charge to the patient's body in the form of an electric shock. The electric shock can be delivered in one or more pulses.

In particular, FIG. 1 also depicts components of a WCD system made according to embodiments. One such component is a support structure 170 that is wearable by ambulatory patient 82. Accordingly, support structure 170 is configured to be worn by ambulatory patient 82 for at least several hours per day, and for at least several days, even a few months or longer. It will be understood that support structure 170 is shown only generically in FIG. 1, and in fact partly conceptually. FIG. 1 is provided merely to illustrate concepts about support structure 170 and is not to be construed as limiting how support structure 170 is implemented, or how it is worn.

Support structure 170 can be implemented in many different ways. For example, it can be implemented in a single component or a combination of multiple components. In embodiments, support structure 170 could include a vest, a half-vest, a garment, etc. In such embodiments such items can be worn similarly to analogous articles of clothing. In embodiments, support structure 170 could include a harness, one or more belts or straps, etc. In such embodiments, such items can be worn by the patient around the torso, hips, over the shoulder, etc. In embodiments, support structure 170 can include a container or housing, which can even be waterproof. In such embodiments, the support structure can be worn by being attached to the patient's body by adhesive material, for example as shown and described in U.S. Pat. No. 8,024,037. Support structure 170 can even be implemented as described for the support structure of U.S. patent application Ser. No. 16/721,506 published as US 2020/0222707 A1, which is incorporated herein by reference. Of course, in such embodiments, the person skilled in the art will recognize that additional components of the WCD system can be in the housing of a support structure instead of being attached externally to the support structure, for example as described in the US2017/0056682 document. There can be other examples.

FIG. 1 generally illustrates a sample external defibrillator 100. As described in more detail later, some aspects of external defibrillator 100 include a housing and an energy storage module within the housing. As such, in the context of a WCD system, defibrillator 100 is sometimes called a main electronics module. The energy storage module can be configured to store an electrical charge. Other components can cause at least some of the stored electrical charge to be discharged via electrodes through the patient, so as to deliver one or more defibrillation shocks through the patient.

FIG. 1 also shows sample defibrillation electrodes 104, 108, which are coupled to external defibrillator 100 via electrode leads 105. Defibrillation electrodes 104, 108 can be configured to be worn by patient 82 in a number of ways. For instance, defibrillator 100 and defibrillation electrodes 104, 108 can be coupled to support structure 170, directly or indirectly. In other words, support structure 170 can be configured to be worn by ambulatory patient 82 so as to maintain at least one of electrodes 104, 108 on the body of ambulatory patient 82, while patient 82 is moving around, etc. The electrode can be thus maintained on the body by being attached to the skin of patient 82, simply pressed against the skin directly or through garments, etc. In some embodiments the electrode is not necessarily pressed against the skin, but becomes biased that way upon sensing a condition that could merit intervention by the WCD system. In addition, many of the components of defibrillator 100 can be considered coupled to support structure 170 directly, or indirectly via at least one of defibrillation electrodes 104, 108.

When defibrillation electrodes 104, 108 make good electrical contact with the body of patient 82, defibrillator 100 can administer, via electrodes 104, 108, a brief, strong electric pulse 111 through the body. Accordingly, proper fitment of the WCD system affects the quality of the electrical contact and hence the quality of the therapy. Pulse 111 is also known as shock, defibrillation shock, therapy, electrotherapy, therapy shock, etc. Pulse 111 is intended to go through and restart heart 85, in an effort to save the life of patient 82. Pulse 111 can further include one or more pacing pulses of lesser magnitude to simply pace heart 85 if needed, and so on.

Defibrillators typically determine whether to defibrillate or not based on an ECG signal of the patient. However, external defibrillator 100 may initiate defibrillation, or hold-off defibrillation, based on a variety of inputs, with the ECG signal merely being one of these inputs. It should be noted that a proper fitment of the WCD system affects the quality of the ECG signal because an ill-fitted WCD system may impact or degrade how well ECG electrodes adhere to the patient 82. A poor connection between the ECG electrodes and the patient 82 impacts how much noise is on the ECG signal and therefore decisions regarding therapy. For this and other reasons, the fitment of the WCD system impacts how well the WCD system performs and, thereby, the effectiveness of the WCD system as a life-saving medical device.

A WCD system according to embodiments can obtain data from patient 82. For collecting such data, the WCD system may optionally include at least an outside monitoring device 180. Device 180 is called an “outside” device because it could be provided as a standalone device, for example not within the housing of defibrillator 100. Device 180 can be configured to sense or monitor at least one local parameter. A local parameter can be a parameter of patient 82, or a parameter of the WCD system, or a parameter of the environment, as will be described later in this document.

For some of these parameters, device 180 may include one or more sensors or transducers. Each one of such sensors can be configured to sense a parameter of patient 82, and to render an input responsive to the sensed parameter. In some embodiments the input is quantitative, such as values of a sensed parameter; in other embodiments the input is qualitative, such as informing whether or not a threshold is crossed, and so on. Sometimes these inputs about patient 82 are also referred to herein as physiological inputs and patient inputs. In embodiments, a sensor can be construed more broadly, as encompassing many individual sensors.

Optionally, device 180 is physically coupled to support structure 170. In addition, device 180 may be communicatively coupled with other components that are coupled to support structure 170. Such communication can be implemented by a communication module, as will be deemed applicable by a person skilled in the art in view of this description.

In embodiments, one or more of the components of the shown WCD system may be customized for patient 82. This customization may include a number of aspects. For instance, support structure 170 can be fitted to the body of patient 82. For another instance, baseline physiological parameters of patient 82 can be measured, such as the heart rate of patient 82 while resting, while walking, motion detector outputs while walking, etc. The measured values of such baseline physiological parameters can be used to customize the WCD system, in order to make its diagnoses more accurate, since patients' bodies differ from one another. Of course, such parameter values can be stored in a memory of the WCD system, and so on. Moreover, a programming interface can be made according to embodiments, which receives such measured values of baseline physiological parameters. Such a programming interface may input automatically in the WCD system these, along with other data.

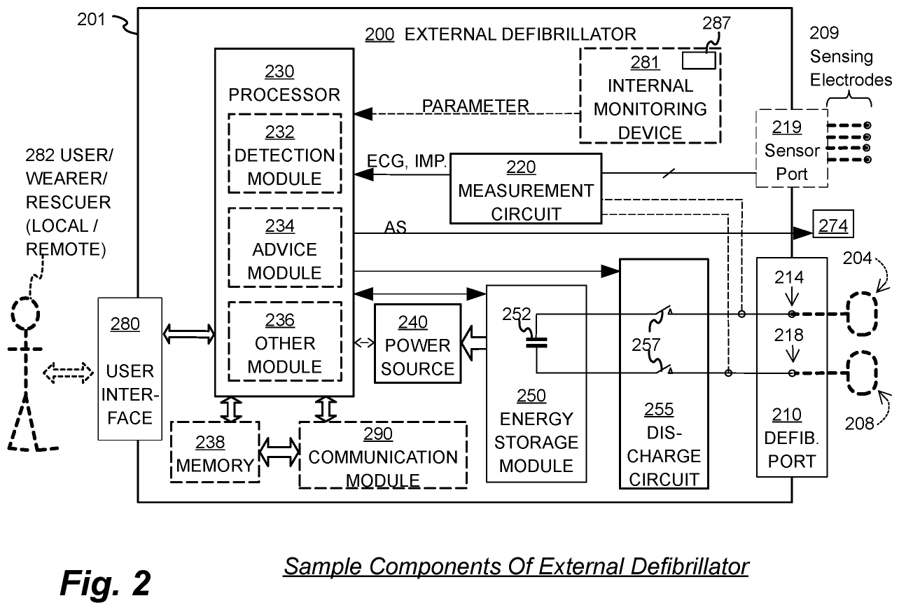

FIG. 2 is another functional diagram showing components of an external defibrillator 200, made according to embodiments. These components can be, for example, included in external defibrillator 100 of FIG. 1. The components shown in FIG. 2 can be provided in a housing 201, which may also be referred to as casing 201.

External defibrillator 200 is intended for a patient who would be wearing it, such as ambulatory patient 82 of FIG. 1. Defibrillator 200 may further include a user interface 280 for a user 282. User 282 can be patient 82, also known as wearer 82. Or, user 282 can be a local rescuer at the scene, such as a bystander who might help, or a trained person. Or, user 282 might be a remotely located trained caregiver in communication with the WCD system.

User interface 280 can be made in a number of ways. User interface 280 may include output devices, which can be visual, audible or tactile, for communicating to a user by outputting images, sounds or vibrations. Images, sounds, vibrations, and anything that can be perceived by user 282 can also be called human-perceptible indications (HPIs). There are many examples of output devices. For example, an output device can be a light, or a screen to display what is sensed, detected and/or measured, and provide visual feedback to rescuer 282 for their resuscitation attempts, and so on. Another output device can be a speaker, which can be configured to issue voice prompts, beeps, loud alarm sounds and/or words to warn bystanders, etc.

User interface 280 may further include input devices for receiving inputs from users. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock.

Defibrillator 200 may include an internal monitoring device 281. Device 281 is called an “internal” device because it is incorporated within housing 201. Monitoring device 281 can sense or monitor patient parameters such as patient physiological parameters, system parameters and/or environmental parameters, all of which can be called patient data. In other words, internal monitoring device 281 can be complementary or an alternative to outside monitoring device 180 of FIG. 1. Allocating which of the parameters are to be monitored by which of monitoring devices 180, 281 can be done according to design considerations. Device 281 may include one or more sensors, as also described elsewhere in this document.

Patient parameters may include patient physiological parameters. Patient physiological parameters may include, for example and without limitation, those physiological parameters that can be of any help in detecting by the WCD system whether or not the patient is in need of a shock or other intervention or assistance. Patient physiological parameters may also optionally include the patient's medical history, event history and so on. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, monitoring devices 180, 281 may include one or more sensors configured to acquire patient physiological signals. Examples of such sensors or transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter is a trend that can be detected in a monitored physiological parameter of patient 282. A trend can be detected by comparing values of parameters at different times over short and long terms. Parameters whose detected trends can particularly help a cardiac rehabilitation program include: (a) cardiac function (e.g. ejection fraction, stroke volume, cardiac output, etc.); (b) heart rate variability at rest or during exercise; (c) heart rate profile during exercise and measurement of activity vigor, such as from the profile of an accelerometer signal and informed from adaptive rate pacemaker technology; (d) heart rate trending; (e) perfusion, such as from SpO2, CO2, or other parameters such as those mentioned above, (f) respiratory function, respiratory rate, etc.; (g) motion, level of activity; and so on. Once a trend is detected, it can be stored and/or reported via a communication link, along perhaps with a warning if warranted. From the report, a physician monitoring the progress of patient 282 will know about a condition that is either not improving or deteriorating.

Patient state parameters include recorded aspects of patient 282, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WCD system made according to embodiments may thus include a motion detector. In embodiments, a motion detector can be implemented within monitoring device 180 or monitoring device 281. Such a motion detector can be made in many ways as is known in the art, for example by using an accelerometer. In this example, a motion detector 287 is implemented within monitoring device 281. A motion detector of a WCD system according to embodiments can be configured to detect a motion event. A motion event can be defined as is convenient, for example a change in motion from a baseline motion or rest, etc. In such cases, a sensed patient parameter is motion.

System parameters of a WCD system can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if monitoring device 180 or 281 includes a GPS location sensor as per the above, and if it is presumed that the patient is wearing the WCD system.

Defibrillator 200 typically includes a defibrillation port 210, which can be a socket in housing 201. Defibrillation port 210 includes electrical nodes 214, 218. Leads of defibrillation electrodes 204, 208, such as leads 105 of FIG. 1, can be plugged into defibrillation port 210, so as to make electrical contact with nodes 214, 218, respectively. It is also possible that defibrillation electrodes 204, 208 are connected continuously to defibrillation port 210, instead. Either way, defibrillation port 210 can be used for guiding, via electrodes, to the wearer at least some of the electrical charge that has been stored in an energy storage module 250 that is described more fully later in this document. The electric charge will be the shock for defibrillation, pacing, and so on.

Defibrillator 200 may optionally also have a sensor port 219 in housing 201, which is also sometimes known as an ECG port. Sensor port 219 can be adapted for plugging in sensing electrodes 209, which are also known as ECG electrodes and ECG leads. It is also possible that sensing electrodes 209 can be connected continuously to sensor port 219, instead. Sensing electrodes 209 are types of transducers that can help sense an ECG signal, e.g., a 12 lead signal, or a signal from a different number of leads, especially if they make good electrical contact with the body of the patient and in particular with the skin of the patient. As with defibrillation electrodes 204, 208, the support structure can be configured to be worn by patient 282 so as to maintain sensing electrodes 209 on a body of patient 282. For example, sensing electrodes 209 can be attached to the inside of support structure 170 for making good electrical contact with the patient, similarly with defibrillation electrodes 204, 208.

Optionally a WCD system according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel, so that it does not flow away, after being deployed, from the location it is released near the electrode. The fluid can be used for both defibrillation electrodes 204, 208, and for sensing electrodes 209.

The fluid may be initially stored in a fluid reservoir, not shown in FIG. 2. Such a fluid reservoir can be coupled to the support structure. In addition, a WCD system according to embodiments further includes a fluid deploying mechanism 274. Fluid deploying mechanism 274 can be configured to cause at least some of the fluid to be released from the reservoir and be deployed near one or both of the patient locations to which electrodes 204, 208 are configured to be attached to the patient. In some embodiments, fluid deploying mechanism 274 is activated prior to the electrical discharge responsive to receiving activation signal AS from a processor 230, which is described more fully later in this document.

In some embodiments, defibrillator 200 also includes a measurement circuit 220, as one or more of its working together with its sensors or transducers. Measurement circuit 220 senses one or more electrical physiological signals of the patient from sensor port 219, if provided. Even if defibrillator 200 lacks sensor port 219, measurement circuit 220 may optionally obtain physiological signals through nodes 214, 218 instead, when defibrillation electrodes 204, 208 are attached to the patient. In these cases, the input reflects an ECG measurement. The patient parameter can be an ECG, which can be sensed as a voltage difference between electrodes 204, 208. In addition, the patient parameter can be an impedance, which can be sensed between electrodes 204, 208 and/or between the connections of sensor port 219 considered pairwise. Sensing the impedance can be useful for detecting, among other things, whether these electrodes 204, 208 and/or sensing electrodes 209 are not making good electrical contact with the patient's body. These patient physiological signals may be sensed when available. Measurement circuit 220 can then render or generate information about them as inputs, data, other signals, etc. As such, measurement circuit 220 can be configured to render a patient input responsive to a patient parameter sensed by a sensor. In some embodiments, measurement circuit 220 can be configured to render a patient input, such as values of an ECG signal, responsive to the ECG signal sensed by sensing electrodes 209. More strictly speaking, the information rendered by measurement circuit 220 is output from it, but this information can be called an input because it is received as an input by a subsequent device or functionality.

Defibrillator 200 also includes a processor 230. Processor 230 may be implemented in a number of ways in various embodiments. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 may include, or have access to, a non-transitory storage medium, such as memory 238 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

Processor 230 can be considered to have a number of modules. One such module can be a detection module 232. Detection module 232 can include a Ventricular Fibrillation (VF) detector. The patient's sensed ECG from measurement circuit 220, which can be available as inputs, data that reflect values, or values of other signals, may be used by the VF detector to determine whether the patient is experiencing VF. Detecting VF is useful, because VF typically results in SCA Detection module 232 can also include a Ventricular Tachycardia (VT) detector, and so on.

Another such module in processor 230 can be an advice module 234, which generates advice for what to do. The advice can be based on outputs of detection module 232. There can be many types of advice according to embodiments. In some embodiments, the advice is a shock/no shock determination that processor 230 can make, for example via advice module 234. The shock/no shock determination can be made by executing a stored Shock Advisory Algorithm. A Shock Advisory Algorithm can make a shock/no shock determination from one or more ECG signals that are captured according to embodiments and determine whether or not a shock criterion is met. The determination can be made from a rhythm analysis of the captured ECG signal or otherwise.

In some embodiments, when the determination is to shock, an electrical charge is delivered to the patient. Delivering the electrical charge is also known as discharging and shocking the patient. As mentioned above, such can be for defibrillation, pacing, and so on.

In ideal conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in U.S. patent application Ser. No. 16/037,990, filed on Jul. 17, 2018 and since published as US 2019/0030351 A1, and also in U.S. patent application Ser. No. 16/038,007, filed on Jul. 17, 2018 and since published as US 2019/0030352 A1, both by the same applicant and incorporated herein by reference.

Processor 230 can include additional modules, such as other module 236, for other functions. In addition, if internal monitoring device 281 is indeed provided, processor 230 may receive its inputs, etc.

Defibrillator 200 optionally further includes a memory 238, which can work together with processor 230. Memory 238 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 may be able to read and execute. More particularly, the programs can include sets of instructions in the form of code, which processor 230 may be able to execute upon reading. The programs may also include other information such as configuration data, profiles, scheduling etc. that can be acted on by the instructions. Executing is performed by physical manipulations of physical quantities, and may result in functions, operations, processes, acts, actions and/or methods to be performed, and/or the processor to cause other devices or components or blocks to perform such functions, operations, processes, acts, actions and/or methods. The programs can be operational for the inherent needs of processor 230, and can also include protocols and ways that decisions can be made by advice module 234. In addition, memory 238 can store prompts for user 282, if this user is a local rescuer. Moreover, memory 238 can store data. This data can include patient data, system data and environmental data, for example as learned by internal monitoring device 281 and outside monitoring device 180. The data can be stored in memory 238 before it is transmitted out of defibrillator 200, or be stored there after it is received by defibrillator 200.

Defibrillator 200 can optionally include a communication module 290, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, communication module 290 may transmit wirelessly, e.g., on a daily basis, heart rate, respiratory rate, and other vital signs data to a server accessible over the internet, for instance as described in US 20140043149. This data can be analyzed directly by the patient's physician and can also be analyzed automatically by algorithms designed to detect a developing illness and then notify medical personnel via text, email, phone, etc. Module 290 may also include such interconnected sub-components as may be deemed necessary by a person skilled in the art, for example an antenna, portions of a processor, supporting electronics, outlet for a telephone or a network cable, etc.

Defibrillator 200 may also include a power source 240. To enable portability of defibrillator 200, power source 240 typically includes a battery. Such a battery is typically implemented as a battery pack, which can be rechargeable or not. Sometimes a combination is used of rechargeable and non-rechargeable battery packs. Other embodiments of power source 240 can include an AC power override, for where AC power will be available, an energy-storing capacitor, and so on. Appropriate components may be included to provide for charging or replacing power source 240. In some embodiments, power source 240 is controlled and/or monitored by processor 230.

Defibrillator 200 may additionally include an energy storage module 250. Energy storage module 250 can be coupled to the support structure of the WCD system, for example either directly or via the electrodes and their leads. Module 250 is where some electrical energy can be stored temporarily in the form of an electrical charge, when preparing it for discharge to administer a shock. In embodiments, module 250 can be charged from power source 240 to the desired amount of energy, as controlled by processor 230. In typical implementations, module 250 includes a capacitor 252, which can be a single capacitor or a system of capacitors, and so on. In some embodiments, energy storage module 250 includes a device that exhibits high power density, such as an ultracapacitor. As described above, capacitor 252 can store the energy in the form of an electrical charge, for delivering to the patient.

A decision to shock can be made responsive to the shock criterion being met, as per the above-mentioned determination. When the decision is to shock, processor 230 can be configured to cause at least some or all of the electrical charge stored in module 250 to be discharged through patient 82 while the support structure is worn by patient 82, so as to deliver a shock 111 to patient 82.

For causing the discharge, defibrillator 200 moreover includes a discharge circuit 255. When the decision is to shock, processor 230 can be configured to control discharge circuit 255 to discharge through the patient at least some or all of the electrical charge stored in energy storage module 250. Discharging can be to nodes 214, 218, and from there to defibrillation electrodes 204, 208, so as to cause a shock to be delivered to the patient. Circuit 255 can include one or more switches 257. Switches 257 can be made in a number of ways, such as by an H-bridge, and so on. Circuit 255 could also be thus controlled via processor 230, and/or user interface 280. A time waveform of the discharge may be controlled by thus controlling discharge circuit 255. The amount of energy of the discharge can be controlled by how much energy storage module has been charged, and also by how long discharge circuit 255 is controlled to remain open.

Defibrillator 200 can optionally include other components.

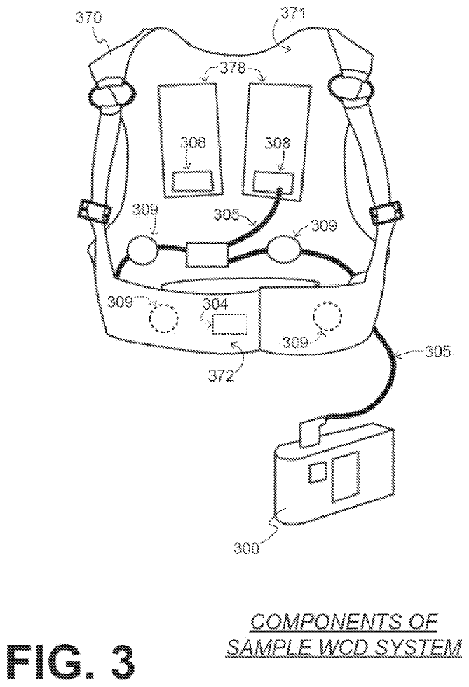

FIG. 3 is another functional diagram illustrative sample embodiments of components of an illustrative WCD system. A support structure 370 includes a vest-like wearable garment. Support structure 370 has a back side 371, and a front side 372 that closes in front of the chest of the patient.

The WCD system of FIG. 3 also includes an external defibrillator 300. FIG. 3 does not show any support for external defibrillator 300, which may be carried in a purse, on a belt, by a strap over the shoulder, and so on. Wires 305 connect external defibrillator 300 to electrodes 304, 308, 309. Of those, electrodes 304, 308 are defibrillation electrodes, and electrodes 309 are ECG sensing electrodes.

Support structure 370 is configured to be worn by the ambulatory patient so as to maintain electrodes 304, 308, 309 on a body of the patient. Indeed, back defibrillation electrodes 308 are maintained in pockets 378. Of course, the inside of pockets 378 can be made with loose netting, so that electrodes 308 can contact the back of the patient, especially with the help of the conductive fluid that has been deployed. In addition, sensing electrodes 309 are maintained in positions that surround the patient's torso, for sensing ECG signals and/or the impedance of the patient. As mentioned above, the proper fitment of the support structure 370 impacts the quality of the signal detected by electrodes 304, 308, 309. It is for this reason that a trained responder should be employed to assist with the fitment of the WCD system and with training of the patient in proper wearing of the WCD system.

ECG signals in a WCD system may include too much electrical noise to be useful. To ameliorate the problem, multiple ECG sensing electrodes 309 are provided, for presenting many options to processor 230. These options are different vectors for sensing the ECG signal, as described now in more detail.

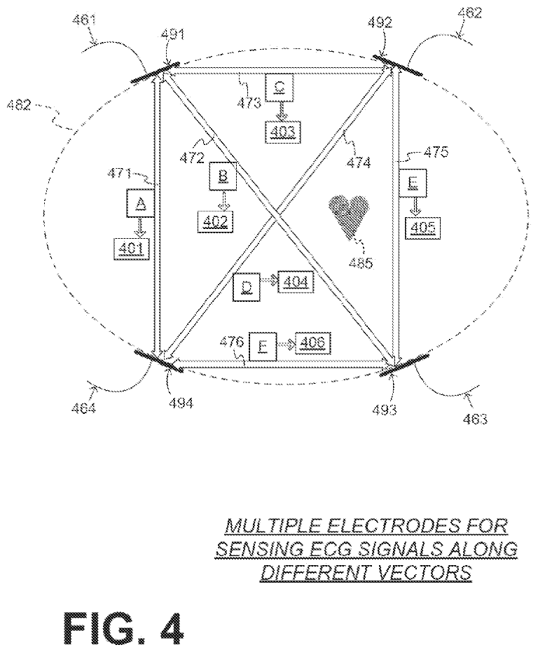

FIG. 4 is a conceptual diagram illustrating how multiple electrodes of a WCD system may be used for sensing ECG signals along different vectors according to embodiments. A section of a patient 482 having a heart 485 is shown. In FIG. 4, patient 482 is viewed from the top, patient 482 is facing downwards, and the plane of FIG. 4 intersects patient 482 at the torso of the patient.

In one embodiment, four ECG sensing electrodes 491, 492, 493, 494 are maintained on the torso of patient 482, and have respective wire leads 461, 462, 463, 464. It will be recognized that electrodes 491, 492, 493, 494 surround the torso, similarly with sensing electrodes 309 in the example of FIG. 3.

Any pair of these four ECG sensing electrodes 491, 492, 493, 494 defines a vector, along which an ECG signal may be sensed and/or measured. As such, electrodes 491, 492, 493, 494 define six vectors 471, 472, 473, 474, 475, 476. FIG. 4 thus illustrates a multi-vector embodiment.

These vectors 471, 472, 473, 474, 475, 476 define channels A, B, C, D, E, F respectively. ECG signals 401, 402, 403, 404, 405, 406 may thus be sensed and/or measured from channels A, B, C, D, E, F, respectively, and in particular from the appropriate pairings of wire leads 461, 462, 463, 464 for each channel.

In FIG. 4 it will be understood that electrodes 491, 492, 493, 494 are drawn as being on the same plane for simplicity and as is preferred, while that is not necessarily the case. Accordingly, vectors 471, 472, 473, 474, 475, 476 are not necessarily on the same plane, either.

In embodiments, in order to make the shock/no-shock determination as correctly as possible, a WCD may assess which of ECG signals 401, 402, 403, 404, 405, 406 is best for rhythm analysis and interpretation. For example, ECG signals that have the most noise may be ignored, discarded, not considered, while leaving the remaining ECG signals as candidates for making the shock/no shock determination.

In other embodiments, the vectors may be aggregated to make a shock/no shock decision, and/or to determine the patient's heart rate and/or QRS widths. For example, in some embodiments the aggregation can be implemented as disclosed in U.S. Pat. No. 9,757,581 issued Sep. 12, 2017 entitled “Wearable Cardioverter Defibrillator Components Making Aggregate Shock/No Shock Determination From Two Or More ECG Signals”, which is incorporated herein by reference.

Embodiments of a system for dispatching responders in accordance with this disclosure will now be described. Generally stated, the several embodiments perform actions and analyses to identify one or more appropriate Patient Service Responders (PSRs). These PSRs assist patients with a medical product fitting, adjusting, positioning, training, and/or any additional follow-ups. In some embodiments, these PSRs are qualified to provide such services, such as by receiving training and periodic renewals of such qualification. In one example, a patient is prescribed a wearable medical device and needs a skilled professional to perform a fitting. In addition, the patient may subsequently need an adjustment of the device. In the case of a WCD, for example, the device may include a garment, sensors, and therapy electrodes, and it may require assembly and adjustment of each of those components. Placement and fitting of other customized elements may also be needed. Proper fit and accurate positioning aids data acquisition, therapy decision making, comfort, and compliance with the prescription.

### BRIEF OVERVIEW OF DISCLOSED TEACHINGS

Embodiments of the system disclosed herein are capable of scheduling, dynamically adjusting in real time, navigating a multitude of conditional criteria and directing high throughput of patients and responders. Embodiments of the systems are further capable of predicting outcomes and planning using machine learning and/or decision trees, for example. These systems can account for and nimbly adjust to a variety of changing attributes, features, or circumstances, including changing geographic parameters, such as responder's travel time, traffic patterns, incidents, or other intervening events, etc. They can quickly engage and dispatch standby responders, if needed.

In addition, in some embodiments the system enables remote teleconferencing (such as using the FaceTime feature provided by the iPhone mobile device), and/or other video connections. A PSR can also hold a call or a teleconference with a patient to guide and/or answer questions without having to physically be present. These tools can be used as pre- or follow-up visits. A conference can be initially held to determine what inventory is additionally needed for the visit and/or further assess the patient size and needs, and also as a way for check-ins post fitting and after a patient begins wearing the device. Such care and follow-up can further improve compliance through the initial period when a patient is still adjusting to the wearable and learning about it. Interactions can be logged and tracked by a responder and/or automatically tracked by the system.

In a further embodiment, a patient, or patient's caregiver, can be provided with personalized training on use and care as well as adjustments and accurate positioning of sensors and therapy modules. Patients, service requestors, or their immediate caregivers, may also need to be trained and learn about resources available to them and those that should be engaged based on escalation in urgency at any given moment.

The system may also be configured to allow an administrator to override and adjust preferences and implement updates when needed. For example, a platform or system administrator can add, delete, edit, switch on or off an attribute or feature, for example, disallowing redundancies or option of one responder responding to two different requisitions for the same time window.

Embodiments of the system can be configured to engage protocols depending on different levels of priority, such as urgent, emergency, and non-emergency events. For example, an event may be urgent where a patient needs attention of high priority, but does not rise to the level of emergency, where, for example, therapy was deployed. In one scenario, an urgent attention, for example, when a product is already worn, is prioritized, or an emergency arises when some type of product alert is triggered. Non-emergency situations can be those cases that don't require quick or immediate intervention and can be scheduled in advance.

### EMBODIMENTS OF A SYSTEM FOR DISPATCHING RESPONDERS

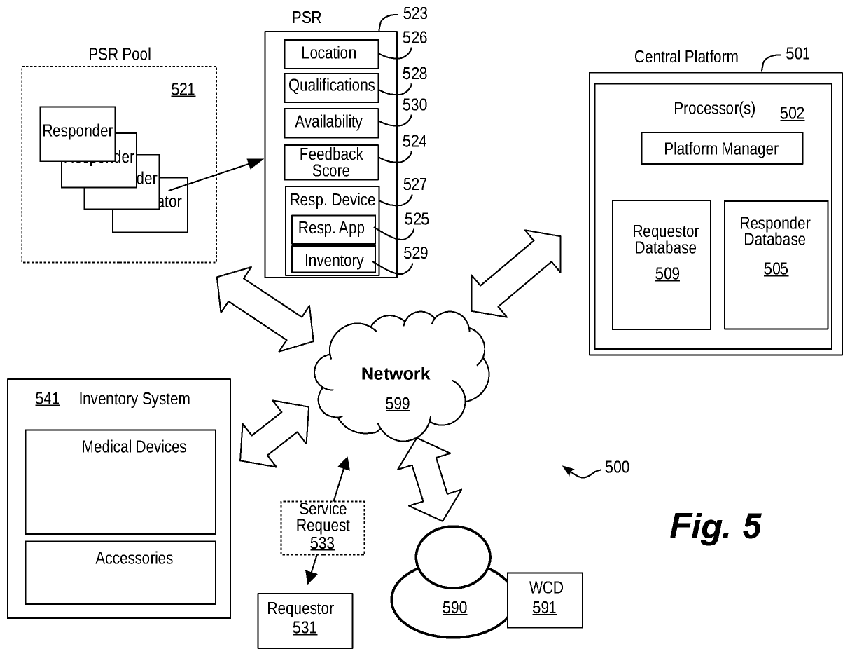

FIG. 5 is a functional block diagram generally illustrating components of one illustrative Resource Management System 500 that implements embodiments of the aforementioned system. The preferred Resource Management System 500 includes a central platform 501, a pool of responders (PSRs) 521, a requestor 531, and an inventory system 541. Each component of the Resource Management System 500 is intended to support a patient 590 who has been prescribed a wearable medical device, such as a WCD 591.

### ILLUSTRATIVE POOL OF RESPONDERS

The illustrative resource management system 500 includes a PSR pool 521. The PSR pool 521 represents a collection of individual patient service responders (PSRs) (sometimes also referred to as a product service responders) (e.g., PSR 523). Each PSR in the PSR pool 521 represents an individual who has received any requisite training necessary to properly fit the wearable medical device (e.g., WCD 591) to the patient 590. For example, each PSR may have received any training necessary to ensure that components (e.g., electrodes) of the WCD 591 are properly affixed to the patient 590 for the most accurate and reliable readings, and for the most effective therapy treatments. Each PSR may also have undergone sufficient training to enable that PSR to similarly train the patient 590 in how to properly wear the WCD 591 for greatest comfort and effectiveness.

Each PSR in the PSR pool 521 has certain associated characteristics which describe that particular PSR. For example, the PSR characteristics may include a location (e.g., Location 526) of the PSR, trainings completed by the PSR and other qualifications (e.g., Qualifications 528), the PSR's current inventories (e.g., PSR Inventory 529), an availability of the PSR (e.g., Availability 530), a feedback score 524 for the PSR, and the like. In addition, characteristics may further include foreign language proficiencies, age, gender, expertise with certain types of device, patient, and/or medical conditions, an ability to lift heavy weight, precautions to be taken, etc. The several characteristics of each PSR may be collected and stored in a responder profile, along with other information.

Each PSR may also maintain his or her own PSR inventory 529, which may include a number of inventory items, such as wearable medical devices and other accessories. Alternatively, the PSR may request that inventory items, such as a WCD, be shipped to the PSR or to an inventory location where the PSR can pick it up, or to a location at which the PSR will provide services to the patient (e.g., the patient's home or workplace).

In certain embodiments, one or more PSRs may also have a responder application (responder app 525). The responder app 525 enables communication between the PSR 523 and at least the Central Platform 501. The responder app 525 may additionally enable communication between the PSR 523 and other elements of the Resource Management System 500, such as the patient 590, a service requestor 531, an inventory system 541, medical personnel supporting the patient 590, and the like. Using the responder app 525, the PSR 523 may indicate an availability for service requests, the PSR's location, the PSR's personal inventory, and other of the PSR's characteristics. Such communication may be initiated manually (e.g., on demand by the PSR 523), or in an automated fashion (e.g., by programmatically issuing such notifications), or some combination of both.

A user interface of the responder app 525 can be configured to provide a sign-up profile application component that enables each PSR to set up and maintain an individual responder profile. The responder profiles may be reviewed upon signup and on a recurring basis, such as to confirm that the PSR's continuing professional qualifications and requisites are kept up to date. The responder profile can include predetermined requisite criteria, such as specific qualifications, certifications, etc., along with other attributes such as responder's age, languages, sex, number of patients to-date, customer satisfaction level scores, comments.

The responder app 525 can be loaded onto an individual stand-alone responder device 527. The responder device 527 may be medical or non-medical, and examples include a special-purpose responder mobile device, a mobile phone, a tablet, or a laptop computer. Other examples are also possible. The responder device 527 can be configured via the responder app 525 to provide a communication portal between the PSR and the Central Platform 501.

The PSR may also engage with the Central Platform 501, such as by using the responder app 525, to obtain any requisite missing or additional information, for example initial measurements or size information, or any other information helpful to perform a fitting or assist the patient.

Once selected and engaged, PSRs may interact with the patient or the requestor directly and may be responsible to ensure the Central Platform 501 is notified about any events, interactions, developments pertaining to a particular product and/or patient. In one particular embodiment, the system may require that communications occur via the Central Platform 501. Alternatively, the Central Platform 501 can be regularly updated by the PSRs post-dispatch.

### SERVICE REQUESTOR AND SERVICE REQUEST

In one embodiment, a service requestor 531 is an individual or entity that initiates a request for a wearable medical device (i.e., a “service request”), such as a wearable cardioverter defibrillator (WCD), to be issued to the patient 590. The service requestor 531 may be medical personnel assigned to the patient 590, a representative of the device supplier or manufacturer, a medical doctor who prescribed the product to the patient 590, a medical or non-medical device (e.g., a mobile phone assigned to the patient 590 or the actual patient 590). The service requestor 531 could send the prescription to the Central Platform 501. Alternatively, the patient 590, having been given the prescription, could act as the service requestor 531 and send a request to the Central Platform 501. Issuing the prescription may initiate a process (such as process 600 described below) for procuring a WCD and also the fitting and training process.

The requestor may use a requestor app 535 configured to interface with and provide requisite information, such as the service request 533, to the Central Platform 501. The requestor app 535 may be used to gather information relevant to the request, which includes the criteria to be used by the Central Platform 501 to match and select PSRs within a categorized pool.

Patient measurements can be initially obtained by the service requestor 531 or, alternatively, the medical doctor or other medical personnel, the patient, the patient's caregiver, or the like if such individual is not the requestor 531, and included in the service request 533. Alternatively, the patient measurements could be obtained by the PSR directly. Patient measurements can also be preliminarily obtained using a size estimator application.

The service request 533 may include other relevant information, such as time and place of fitting. The service request 533 may identify available or preferred time windows and include additional criteria, for example less preferred but available times and location(s).

In a further enhancement, the service request 533 may include a patient's preferences regarding PSR, such as a particular PSR based on gender, for example, for fitting of a wearable cardioverter defibrillator. Similarly, a patient may also request that the PSR be over a certain age, have a particular foreign language fluency, have assisted/trained at least a certain number of patients, have at least a certain customer satisfaction level rating based on feedback, be free of negative feedback or comments, etc. Such additional criteria may be included in the service request 533. Likewise, any of such criteria may be delivered outside of the initial service request 533, such as verbally or through another mechanism.

Other relevant conditions can be noted in the service request 533, such as, for example, that the patient has sleep apnea or that there is a need for monitoring Congestive Heart Failure, etc. The requestor 531 may also indicate criteria such as preferred language, age, gender of the patient, requested gender of a responder who is to fit the WCD to the patient.

### ILLUSTRATIVE INVENTORY SYSTEM

The illustrative Resource Management System 500 also includes an inventory system 541. The inventory system 541 includes one or more inventory databases that identify, and perhaps also describe, medical devices and accessories that are available to the Resource Management System 500. In addition, the inventory system 541 may also identify a location and availability for each of the medical devices and accessories. Each item identified by the inventory system 541 is considered available for use by one or more of the PSRs in the PSR pool 521.

The inventory system 541 is accessible by other elements of the Resource Management System 500, such as the Central Platform 501, using network communication mechanisms, such as a wide area network, a private network connection, or both. The inventory system 541 is configured to receive and respond to requests for inventory information and reservations from other elements of the Resource Management System 500.

Products identified as available by the inventory system 541 may in fact reside in one or more inventory locations, such as warehouses or inventory storage locations. In addition, the inventory system 541 may expose interfaces that enable individual PSRs to advertise the availability of their own individual inventories (e.g., PSR inventory 529). Items identified by the inventory system 541 may be made available either by pick up at various inventory storage locations, or those items may alternatively be shipped directly to a requested location, such as the patient location, to await the PSR to open and apply it.

Inventory may be assessed for product and size availability. Even when initial size and fitting is provided by a PSR, for example as part of the prescription, a PSR upon dispatch may find that a different fit is needed and may use his/her sizing application to further individualize the fit. In one embodiment, if the size or product is not available, a responder is dispatched to obtain a more accurate fitting measurements, and based on these measurements, the right product is ordered. The responder can then via the Central Platform 501 procure the product and have the fitting visit with the patient scheduled. Alternatively, a responder may have an inventory on hand that is suitable to for average range of XS, Small, Medium, Large, XL, or XXL sized male and/or female patients and may simply utilize what's in his/her inventory.

### ILLUSTRATIVE CENTRAL PLATFORM

As shown in FIG. 5, one embodiment of the Central Platform 501 comprises one or more computer processors 502 configured to receive notifications of events, service requests, and/or locations from the requestor 531 (described above). Embodiments of the Central Platform 501 may be used to assist a patient 590 who has been prescribed a medical product and needs to have the product fitted, tailored, or adjusted to the patient or patient's body, and potentially to also provide to the patient advice, guidance, training on the product use and/or maintenance. Such systems can help facilitate optimal service, use, comfort, compliance, data acquisition, and/or treatment, etc., and as such are highly desirable in the field.

As is described in greater detail in the following paragraphs, generally stated, the Central Platform 501 processes received notifications of events, requests, situation updates, and/or locations, and pushes out notifications and receives responses from the selected, remotely located PSRs via a network and via the patient service remote responder user interface application.

The Central Platform 501 includes databases of responder profiles (e.g., Responder Database 505) for each of the PSRs in the PSR pool 521 including their location, time-to-patient arrival estimate, date, or other relevant characteristics. Such information may be used by the Central Platform 501 to dynamically prioritize, score, and match against needs and criteria of the patient. Examples of the information that may be used to compare each PSR with the patient include foreign language proficiencies, age, gender, expertise with certain types of device, patient, and/or medical conditions, ability to lift heavy weight, precautions to be taken, etc.

The Central Platform 501 receives requests (e.g., Service Request 533) for service providers (e.g., PSRs) from requestors (which may be identified in a Requestor Database 509), and endeavors to fill those requests. As part of that request fulfillment, the Central Platform 501 evaluates each request to identify a matching PSR to fulfill the service request 533. Although the term “matching” may be used throughout this disclosure, it should be appreciated that the PSR who is ultimately either selected to fulfill the service or in fact fulfills the service may not necessarily be the “best match”, meaning another PSR may be most ideally qualified to perform the service but other factors may converge to result in a different PSR being selected, such as the unavailability of the most ideally qualified PSR, or the like. In certain embodiments, the Central Platform 501 may provide a requestor with an option to choose between different PSRs or to request a particular PSR that the patient or requestor has interacted with in the past or would like to request for other reasons.

The Central Platform 501 is further programmed to prioritize PSRs to fulfill service requests based on a number of criteria, such as the PSR's feedback rating, qualifications, location, availability, and on-hand inventory. For example, in one embodiment, each PSR may have a feedback score (e.g., Feedback Score 524) that the Central Platform 501 may use to prioritize PSRs. In one embodiment, the Central Platform 501 is further programmed to respond to the requestor with feedback if a specially requested PSR is not available or available for a different time and depending on the requester's reply, offer other options and/or schedule or dispatch another responder.

The Central Platform 501 includes a facility to push out notifications within predetermined parameter(s) (for example, a geographic parameter) to one or more PSRs that are identified as a “match” with a service request. The Central Platform 501 receives any responses to those notifications from the matching PSRs. In such a manner, the Central Platform 501 is operative to first identify potential PSRs that have identified as available to fulfill a service request, and also confirm that availability directly with each such PSR prior to actually scheduling any service conferences (e.g., initial calls, video conferences, in-person visits, follow up visits, etc.).

If a service request or prescription lacks any needed information or presents an ad hoc case, the Central Platform 501 may engage in an iterative information exchange with the requestor to gather such information, such as by the requestor application, email, text, or call, or other means of communication. Based on the information provided by the requestor, the Central Platform 501 can select and send out notifications for service to relevant PSRs within the functions and geographies requested.

The Central Platform 501 may provide to the selected PSR information and directions as to where and/or when the product will be provided or can be picked up from a particular location. In one embodiment, the device pickup location may be optimized along the route to the patient. In certain embodiments, the PSR 523 maintains a separate inventory and may not need to pick up inventory items to fulfill the service request. In other embodiments, the PSR 523 does not maintain a separate inventory and would pick up the necessary inventory items if not otherwise provided.

The Central Platform 501 may also be configured to centrally manage interfacing with and transmitting notifications to a patient service remote responder application. The notifications may be transmitted through various means, such as via wireless links and the Internet.

Progress of the PSR can be monitored from the time of dispatch through completion of a visit and also to further follow-ups. Follow-ups can be performed by the PSR and also monitored when logged in using the responder app, or other communication means, which can be routed via and/or communicated to the Central Platform 501. Follow-ups can be scheduled on a recurring basis or requested as needed. Service providers' accounts can be managed and monitored including monitoring of completion of tasks.

In embodiments, the Resource Management System 500 allows an administrator to override and/or adjust preferences if needed. In some circumstances, when the prescription contains unusual requests, or lacks some requisite information, an administrator (or other service provider) for the Central Platform 501 may assist the requester. Otherwise, the Central Platform 501 can analyze and assign the prescription and select a responder in an automated fashion.

### ILLUSTRATIVE PROCESS FOR DISPATCHING PATIENT SERVICE RESPONDERS (PSR)

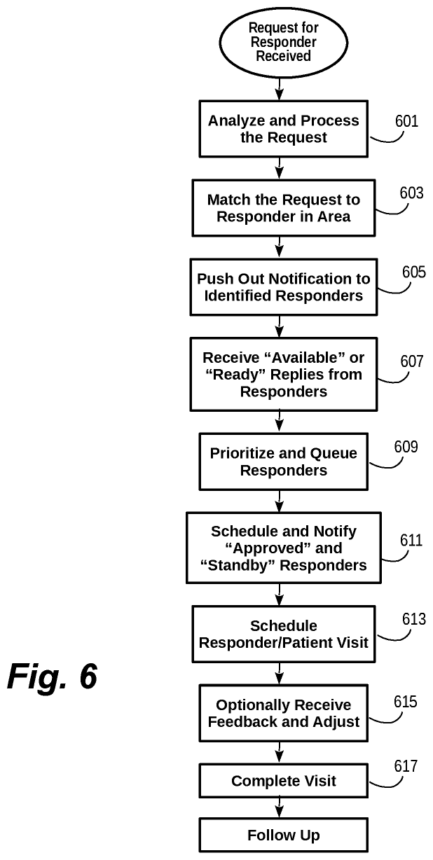

FIG. 6 illustrates an illustrative process 600 that may be performed by one or more of the components of the Resource Management System 500 to implement the teachings of this disclosure. The illustrative process 600 is but one of several alternative processes that may be performed to implement the teachings of this disclosure. Process 600 is detailed here to provide guidance as to the general functions that are provided by each of the components of the Resource Management System 500. It should be noted that such general functions need not be performed by components identified above, and those functions may instead be performed by or distributed among other components of an equivalent Resource Management System.

In one embodiment, the process 600 begins when a Central Platform 501 receives a service request for a PSR from a requesting device. The request can include a service location or patient location, desired service time, qualifications needed to address patient's needs and/or device needs (maintenance, adjustment, repair), and/or inventory (device, garment, accessories, measuring and adjusting tools). The process can be iterative. If no responders are available at the first time slot chosen by a requestor, other time windows can be used to accelerate scheduling.

At step 601, the process 600 analyzes and processes the service request. Embodiments of the Central Platform 501 shown in FIG. 5 can, upon receipt of the service request, analyze the request and categorize it using a decision tree and based on specific requirements, context, and predictive information, such as time of arrival based on traffic patterns.

At step 603, the process 600 reviews the profile information for any PSRs in the area of the patient who may satisfy the request to identify one or more matching PSRs. The Central Platform 501 may identify matching PSRs by also reviewing the PSRs stored criteria and inventory. Still further, the Central Platform 501 may interact with a centralized inventory system to identify whether PSRs may satisfy the service request with inventory that may be made available to those PSRs. The Central Platform 501 can further base its determinations on available inventory vis-a-vis patient's specifications, for example patient size.

In one embodiment, the process 600 may prioritize PSRs to fulfill the service request, at least in part, based on a quality feedback loop by ranking available PSRs based on a feedback score for each PSR. A ranking of PSRs, based at least in part on each PSR's feedback score, may be used to prioritize PSRs in order of preference. The feedback score for each PSR may be reviewed periodically or at each new service request so that the ranking may be dynamically adjusted.

At step 605, the process 600 pushes out notifications to any PSRs identified at step 603. In some embodiments, the Central Platform 501 may directly reach individual PSRs via text message, instant message, phone call, email, or the like. In one embodiment, the process 600 may contact each PSR in order according to any ranking of PSRs, provided that time allows. In addition, the Central Platform 501 may issue notifications to a responder app held by one or more of the potential PSRs. These and other notification mechanisms may be used to notify potential PSRs of the availability of a service request that they may fulfill.

At step 607, the process 600 receives an “available” reply or a “ready” reply from one or more PSRs that were notified at step 605. In one example, one or more potentially matching PSRs that were notified at step 605 returns a reply indicating that PSR's willingness and in fact availability to fulfill the service request. The replies may further include additional information, such as immediate location and current inventory, so that the Central Platform 501 may better prioritize PSRs for dispatch.

At step 609, the process 600 prioritizes and queues the one or more PSRs that replied with availability at step 607. For example, the Central Platform 501 may build a queue and place PSRs on a standby depending on the top ranked PSR's situation or developments. For example, the Central Platform 501 may determine the top match as the PSR who first responds as available for the requested time. However, should a traffic pattern dynamically alter the anticipate schedule, or some other intervening event take place—for example a previous appointment runs later than expected—another PSR from the standby list may be quickly dispatched to provide the service. Some PSRs may also indicate that they are willing to be on a standby, if necessary, etc.

A PSR can be placed in the standby queue based on several factors, such as the location, inventory, skill set, and other personalized and objective criteria of each PSR. A PSR can also be placed on a standby to aid the first selected and dispatched provided in certain cases. For example, if additional equipment is needed upon the first dispatched provider's assessment at the location, or if assistance is needed with heavy lifting, etc.

At step 611, the process 600 schedules at least one “approved” PSR and may additionally schedule “stand by” PSRs. The process 600 also issues notifications to those PSRs that they have been either scheduled or put on standby. Such notification may be provided through any of the aforementioned notification mechanisms (e.g., responder app, text message, instant message, email, or the like).

At step 613, the process 600 schedules a visit (or other initial contact) between the patient and the approved PSR. In one example, the Central Platform 501 may issue a schedule notification (again, using any appropriate notification mechanism) to at least the patient and to the selected PSR. The schedule notification may additionally be transmitted to the requestor (if not the patient) to inform the requestor that the service request is scheduled for fulfillment. Other individuals or entities may also be notified for various reasons. For example, the inventory system 541 may be notified so that it may make any necessary inventory items available. Other examples will also become apparent.

In one enhancement, the schedule notification may include information about the scheduled visit (or other initial contact) such as time and place, and may also include information about the selected PSR, such as the PSR's qualifications and/or characteristics. The schedule notification may even further include personal attributes that the patient and/or requestor may appreciate. In one example, the schedule notification could even include a picture of the PSR, such as to aid with positive identification of the PSR and to informally introduce the PSR to the patient. In this way, the customer experience may be improved with the patient and/or the requestor.

At step 615, the process 600 optionally receives feedback from either the patient, the PSR, or possibly both, about the experience. The feedback could take any one or more of many forms, such as a phone call from the patient with feedback, an automated feedback collection process, a passive website, or the like. In another embodiment, each PSR can be assessed by the requester or the patient, or both, via a survey, comments and a profile score, client/customer satisfaction level, or the like. Such feedback may be added to the PSR's responder profile and used to modify or supplement each PSR's feedback score.

At step 617, the process 600 notes that the requested service has been concluded. Such a notation may include the collection of service-related information, such as any feedback or other criteria gathered about the provision of the service, and any metrics collected during the process of providing the service. Such metrics may be used to further refine the process 600 in subsequent operations.

Other embodiments include combinations and sub-combinations of features described or shown in the drawings herein, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

What is claimed is:

1. A medical product resource management system, comprising:
a medical device configured to selectively provide treatment to a patient, wherein the medical device has a size or type corresponding to the patient;
one or more responder devices associated with one or more responders qualified to assist the patient in using the medical device;
an inventory system including one or more databases configured to identify a plurality of medical devices, including a size or type of each of the plurality of medical devices, available to at least one of the one or more responders; and
a central platform including a database of responder profiles, at least one responder profile associated with a corresponding responder, the responder profile including information that identifies one or more characteristics of the corresponding responder, the one or more characteristics including a qualification of the corresponding responder to assist the patient with the medical device,
wherein the central platform comprises one or more processors configured to:
match a responder of the one or more responders based on a service request received to render assistance to the patient with a medical device, wherein to match the responder, the one or more processors are configured to:
determine availability of the one or more responders based on at least one of the one or more characteristics including a location of the one or more responders; and
receive a reply from at least one matching responder from the available one or more responders via a responder device of the at least one matching responder, in response to a notification communicated to the one or more responders;
responsive to the reply received from the at least one matching responder, verify whether a medical device of the plurality of medical devices from the inventory system is the medical device having the size or type corresponding to the patient;
determine whether the medical device is properly fitted to the patient by the at least one matching responder based on one or more readings acquired from one or more sensors; and
in response to a determination that the medical device is properly fitted to the patient:
enable the medical device to monitor the patient for a medical event, and
upon detecting that the medical event is being experienced by the patient, administer therapy to the patient via the medical device, wherein an effectiveness of the administered therapy is related to an accuracy of a fitment of the medical device on the patient.

2. The medical product resource management system of claim 1, wherein the one or more readings acquired by the one or more sensors are used to adjust the fitment of the medical device.

3. The medical product resource management system of claim 1, wherein the medical device includes a wearable support structure that maintains the medical device in a predefined position on a body of the patient when the patient is wearing the medical device, and wherein the wearable support structure is adjusted based on the accuracy of the fitment of the medical device to ensure the effectiveness of the therapy.

4. The medical product resource management system of claim 1, wherein the medical device comprises a user interface to receive an input from a user to prevent or cease the administration of therapy to the patient.

5. The medical product resource management system of claim 4, wherein the user interface is configured to provide visual feedback to the user for resuscitation attempts.

6. The medical product resource management system of claim 1, wherein the central platform is configured to determine the location of the at least one matching responder based on a geographic proximity to the patient, and wherein the availability of the at least one matching responder is prioritized based on the location of the at least one matching responder and severity of the medical event.

7. The medical product resource management system of claim 1, wherein the medical device is a defibrillator configured to deliver defibrillation therapy.

8. The medical product resource management system of claim 1, wherein the responder device comprises an interface configured to allow the at least one matching responder to communicate with the patient via the central platform to provide training to the patient on how to position and wear the medical device.

9. The medical product resource management system of claim 1, wherein the central platform is configured to receive the service request from a requester to schedule an appointment between the at least one matching responder and the patient.

10. The medical product resource management system of claim 9, wherein the service request includes one or more time slots during which the patient is available for the appointment.

11. The medical product resource management system of claim 9, wherein the service request includes information indicative of physical measurements of the patient.

12. The medical product resource management system of claim 9, wherein the service request includes information indicative of a medical history of the patient.

13. The medical product resource management system of claim 9, wherein the service request includes information indicative of the patient's preferences for the responder.

14. The medical product resource management system of claim 13, wherein the patient's preferences includes one or more of language fluency, gender, and/or experience in providing assistance to use the medical device.

15. A method for managing resources in a medical device provisioning system, the method comprising:
matching a responder of one or more responders based on a service request received to render assistance to a patient with a medical device, wherein the matching comprises:
determining, by a central platform, availability of the one or more responders based on at least one of one or more characteristics including a location of the one or more responders; and
receiving, by the central platform, a reply from at least one matching responder from the available one or more responders via a responder device of the at least one matching responder, in response to a notification communicated to the one or more responders;
responsive to receiving the reply from the at least one matching responder, verifying, by the central platform, whether a medical device of a plurality of medical devices from an inventory system is the medical device having a size or type corresponding to the patient;
determining, by the central platform, whether the medical device is properly fitted to the patient by the at least one matching responder based on one or more readings acquired from one or more sensors; and
in response to a determination that the medical device is properly fitted to the patient:
enabling the medical device to monitor the patient for a medical event, and
upon detecting that the medical event is being experienced by the patient, administering therapy to the patient via the medical device, wherein an effectiveness of the administered therapy is related to an accuracy of a fitment of the medical device on the patient.

16. The method of claim 15, wherein the service request comprises information describing the patient, and wherein the information is used in matching the responder.

17. The method of claim 15, wherein the service request comprises a location of the patient, and wherein the location of the patient is used to assist in matching the responder.

18. The method of claim 15, wherein matching the responder further comprises selecting a service responder with a profile that has at least one of the one or more characteristics that corresponds to a similar preferred characteristic in the service request.

19. The method of claim 15, wherein the one or more sensors are configured to detect one or more physiological parameters of the patient, and wherein the one or more readings from the one or more sensors are used to adjust the fitment of the medical device.

20. The method of claim 15, further comprising receiving the service request from a requester to schedule an appointment between the at least one matching responder and the patient.
